(19) United States
(12) Patent Application Publication
(10) Pub. No.: US 2025/0135138 A1
(43) Pub. Date: May 1, 2025

(54) ARTICULATING STYLET

(71) Applicant: SmartAirway LLC, Torrance, CA (US)

(72) Inventor: Richard Rutgers, Venice, CA (US)

(73) Assignee: SmartAirway LLC, Torrance, CA (US)

(21) Appl. No.: 18/926,117

(22) Filed: Oct. 24, 2024

Related U.S. Application Data
(60) Provisional application No. 63/593,620, filed on Oct. 27, 2023.

(51) Int. Cl. A61M 16/04; A61M 25/01

(52) U.S. Cl. CPC A61M 16/0488; A61M 25/0102

(57) ABSTRACT

The present invention is directed to a new and novel articulating stylet medical device that facilitates endotracheal intubation. During an intubation procedure, the present device uniquely guides a distal portion of a standard, non-proprietary endotracheal tube (ETT) mounted thereover within the narrow confines of a patient's pharynx under visual control via a laryngoscope and then through the larynx and then in to the trachea. Thus, the articulating stylet of the present invention facilitates a faster and safer intubation procedures compared to prior art stylet medical devices.

### CROSS REFERENCE TO RELATED APPLICATION

This patent document claims priority to earlier filed U.S. Provisional Patent Application Ser. No. 63/593,620, filed Oct. 27, 2023, the entire contents of which are incorporated herein by reference.

### BACKGROUND OF THE INVENTION

The present invention relates generally to the medical procedure of endotracheal intubation and an endotracheal tube (ETT) used by a medical professional as part of that intubation procedure. The medical procedure of endotracheal intubation continues to be technically difficult, with associated patient morbidity, mortality and economic losses. Therefore, there is a need to bridge the technology gap associated with this procedure.

By way of background, in the field of medicine, there is the need in surgery and in medical emergencies to establish and maintain a patent airway to ensure adequate exchange of oxygen and carbon dioxide, known as ventilation. A tracheal tube, which is a catheter or conduit, is inserted into the trachea, via the mouth or nose for this purpose, in a procedure called endotracheal intubation. The most common type of ETT is made of polyvinyl chloride, may be cuffed or uncuffed, may be reinforced or not, and may have other features. Further details of ETTs need not be provided herein as these medical devices are very well known in the medical industry.

A known problem with ETTs and the intubation procedure is the ability to navigate through the pharynx, past the vocal cords and into the larynx while avoiding routing the ETT into the patient's esophagus. This navigation and positioning of the ETT can be challenging because a patient's anatomy is not linear and is subject to considerable interpatient variability. Moreover, a patient's pharynx, larynx and trachea are fragile and can be easily damaged by a foreign object therein, such as an ETT or laryngoscope. During emergencies, time is of the essence to provide life-saving oxygen thereby requiring fast, easy and accurate routing of the ETT into the trachea.

ETTs have some flexibility but require some structural rigidity as well, so as not to collapse during gas exchange. Thus, for the above reasons, the steering of an ETT into a patient's trachea requires extreme care and it can be challenging to direct and steer the ETT within the patient without causing injury.

In the prior art, there have been efforts to help steer or guide the ETT into the patient for fast and safe intubation. For example, tracheal tube introducers (Eschmann introducers or “bougies”) are well known devices made of a stiffer material but substantially narrower than an ETT, in which the introducer is first advanced through the pharynx and into the trachea. Then, the ETT is pushed or “railroaded” over the introducer to place the ETT in the trachea. Then the introducer is removed, leaving the ETT in place.

Stylets are also very common to assist in the intubation process. A stylet is a rigid or malleable rod or wire, commonly made of metal, where the malleable stylet is placed inside the endotracheal tube and the two are bent together, the flexible ETT assuming the shape of the stiffer, malleable stylet inside it, the resulting shape theoretically corresponding to the individual patient's airway anatomy. Thus, the stylet is manipulated to estimate the shape of the anatomy of the patient, with the ETT mounted thereon, and is then inserted into the patient. If the shape of the stylet and ETT combination chosen does not conform to the individual patient's anatomy, the ETT and stylet are removed together, reshaped to a more favorable configuration, and the procedure repeated.

Still further, articulating stylet devices are known which reside in the ETT during intubation, like all stylets, but have portions, namely the tip, that articulate while inside the ETT during the intubation to, in turn, articulate the distal tip of the overlying ETT itself to guide the ETT during the intubation procedure. However, these known guided stylet devices are difficult to control with precision, complicated in construction and expensive.

Therefore, there is a need for an intubation assistance device to make the intubation procedure safer and faster.

There is also a need for an intubation assistance device that can accurately and quickly shape an ETT to the patient's anatomy for a successful intubation on the first attempt since repeated attempt are strongly associated with patient injury.

There is a further need for an intubation assistance device in the form of a stylet that can articulate an on-board ETT mounted thereover during the intubation process to simultaneously shape the ETT and steer it for faster intubation.

There is also a need for a an articulating stylet with a controllable tip that can deflect in at least two directions to, in turn, articulate the tip of an ETT mounted thereon while it is being steered through the pharynx and into the patient's trachea.

There is yet another need for an articulating stylet for an ETT that helps reduce the time required to perform intubation.

There is a further need to provide an articulating stylet with precise control of the stylet tip.

There is yet another need for an articulating stylet that is inexpensive and easy to manufacture.

### SUMMARY OF THE INVENTION

The present invention preserves the advantages of prior art articulating stylets for use with ETTs during an intubation. In addition, the present invention provides new advantages not found in currently available devices and methods and overcomes many disadvantages of such currently available devices and methods.

The articulating stylet of the present invention is a medical device used to facilitate endotracheal intubation and addresses the adverse events that commonly befall patients during the procedure. The present device is uniquely capable of dynamically steering the distal portion of an onboarded, standard, non-proprietary endotracheal tube (ETT) within the narrow confines of a patient's pharynx while under constant visual control. When the vocal cords are visualized, the precise angulation afforded by the articulating tip is utilized to direct the ETT carefully into the larynx and trachea.

The present invention includes a hollow outer tube which is in continuity with a handle portion. Within the outer tube is a lumen, within which resides a central shaft running longitudinally the entire length of the hollow tube and which is in continuity with a rotary lever that employs mechanical advantage. The most distal portion of the central shaft is firmly affixed to the outer tube's most distal end. In accordance with the present invention, this central shaft core may have several configurations, including an embodiment in which a malleable wire core is attached distally to a short section of plastic shaft which in turn is attached most distally to the outer tube. The central shaft core may also be a plastic rod with malleable wire in-molded only in a restricted section of the shaft, or the wire shaft may incorporate plastic stabilizers within the hollow tube's lumen.

The central shaft core is attached proximally via an adjustment (attaching) mechanism, such as a clevis or other such structure to a preferably rotary lever which employs mechanical advantage to the wire core. Gripping the handle, which is in continuity with the outer tube, with the palm and fourth and fifth fingers, and applying opposing hand-grip force to the lever, which is in continuity with the central shaft, such as with the thumb, index and third fingers, force is transferred with mechanical advantage to the wire shaft core and reciprocally to the handle and outer tube or sheath, resulting in deformation of the tube/core structure of the device of the present invention. Tensile force applied to the central core via the lever shortens the core relative to the outer tube and selectively results in the distal tip moving superiorly. Compressive force applied to the core results in lengthening of the core relative to the outer tube and the distal tip will move inferiorly. The result is a reciprocal, or push-pull mechanism in which force is transmitted via both “limbs” of the device (the core and the outer tube). To restrict this deformation to only the most distal portion of the device, such as the tip, and to favor only deflection within the sagittal plane, kerfs or tessellations are placed on the superior surface of the distal outer tube and inferior surface of the distal central shaft. In another embodiment the kerfs are placed on the superior surface of the core and the inferior surface of the outer sheath. When the deforming force applied to the device's tip is sufficient to overcome the durometer of the onboarded, overlying ETT, the ETT will deflect in accordance with the force applied. The ETT itself has potential energy incorporated in its forming and will recoil to nearly its original position when the deforming force is lessened or stops. Thus, actuation of the lever causes the wire shaft core to slide relative to the outer tube to achieve the desired deflection of the tip of the ETT in the sagittal plane. The lever employed in the present embodiment may be provided in different configurations and is preferably of a “capstan” or “cockscomb” shape and/or may have gripping surfaces any desired ergonomic position or positions.

It should be noted that the current embodiment mimics the action of a bronchoscope in that inferior movement of the lever causes upward or anterior movement of the tip and ETT, but this can easily be reconfigured so that inferior movement of the lever will cause inferior or downward movement of the tip.

Therefore, an object of the present invention is to provide an intubation assistance device to make the intubation procedure safer and faster.

A further object of the present invention is to provide an intubation assistance device that can accurately and quickly shape an ETT, in situ, under constant visual control according to the patient's unique anatomy for a successful intubation on the first attempt and without adverse events.

Yet another object of the present invention is to provide an intubation assistance device in the form of a stylet that can articulate with an ETT mounted thereover during the intubation process to simultaneously shape the ETT and steer it for faster intubation.

Another object of the present invention is to provide an articulating stylet with a controllable tip that deflects in at least two directions to, in turn, articulate the tip of an ETT mounted thereon all while it is being steered into the patient's trachea.

Yet another object of the present invention is to provide an articulating stylet for an ETT that helps reduce the time it takes to perform an intubation.

Still a further object of the present invention is to provide an articulating stylet with precise control of the stylet tip.

Another object of the present invention is to provide an articulating stylet that is inexpensive and easy to manufacture yet still effective in guiding an ETT during intubation.

Another object is to provide a single-use device so as to prevent cross-contamination between patients in a time of respiratory pandemics.

### BRIEF DESCRIPTION OF THE DRAWING FIGURES

The novel features which are characteristic of the present invention are set forth in the appended claims. However, the invention's preferred embodiments, together with further objects and attendant advantages, will be best understood by reference to the following detailed description taken in connection with the accompanying drawings in which:

FIG. 1 is a perspective view of the articulating stylet of the present invention;

FIG. 2 is a front, partial cross-sectional view of the stylet of FIG. 1;

FIG. 3 is a side view of the stylet of FIG. 1;

FIG. 4 is rear view of the stylet of FIG. 1;

FIG. 5 is a front perspective view of the wire core used in the stylet of FIG. 1;

FIG. 6 is a rear perspective view of the wire core of FIG. 5;

FIG. 7 is a side elevational view of the wire core of FIG. 5;

FIG. 8 is a close-up perspective view of the eyelet proximal end of the wire core of FIG. 5;

FIG. 9 is perspective view of the inner spine used in the stylet of FIG. 1 of the present invention;

FIG. 10 is a side elevational view of the inner spine of FIG. 9;

FIG. 11 is a top view of the inner spine of FIG. 9;

FIG. 12 is a cross-section view through the line 12-12 of FIG. 11;

FIG. 13 is a perspective view of the outer sleeve used in the stylet of FIG. 1 of the present invention;

FIG. 14 is a close-up perspective view of the outer sleeve of FIG. 13;

FIG. 15 is a side elevational view of the outer sleeve of FIG. 13;

FIG. 16 is a close-up end perspective view of the distal end of the outer sleeve of FIG. 13;

FIG. 17 is a side elevational view of the lever of the handle, mounted on a pivot shroud, in a upward position corresponding to an upwardmost position of the tip of the stylet of the present invention;

FIG. 18 is a side elevational view of the lever of the handle in a neutral position corresponding to a neutral position of the tip of the stylet of the present invention;

FIG. 19 is a side elevational view of the lever of the handle in a downward position corresponding to a downwardmost position of the tip of the stylet of the present invention;

FIG. 20 is a rear perspective view of the lever of the handle of FIG. 17;

FIG. 21 is a rear view of the lever of FIG. 17;

FIG. 22 is a side elevational view of the lever of the lever of FIG. 17;

FIG. 23 is a front elevational view of the lever of the lever of FIG. 17;

FIG. 24 is a front perspective view of the pivot shroud of the stylet of the present invention;

FIG. 25 is a rear perspective view of the pivot shroud of FIG. 24;

FIG. 26 is a front view of the pivot shroud of FIG. 24;

FIG. 27 is a rear view of the pivot shroud of FIG. 24;

FIG. 28 is a side elevational view of the pivot shroud of FIG. 24;

FIG. 29 is a perspective view of the pivot shroud and wire core with portions of the pivot shroud shown in phantom for illustration purposes;

FIG. 30 is a perspective view of the pivot shroud and wire core with portions of the pivot shroud shown in phantom for illustration purposes with lever of the handle;

FIG. 31 is a side view of the pivot shroud, wire core and partial cut-away of lever of the handle in a neutral position;

FIG. 32 is a side view of the pivot shroud, wire core and partial cut-away of lever of the handle in an upward most position;

FIG. 33 shows the wire core;

FIG. 34 shows the inner spine attached to the distal end of the wire core;

FIG. 35 shows the outer sleeve residing over the inner spine and wire core to provide a completely assembled stylet of the present invention;

FIG. 36 shows a endotracheal tube mounted over the stylet of the present invention in preparation for use in an intubation procedure;

FIG. 37 shows a front elevational view of an endotracheal tube mounted over the stylet of the present invention;

FIG. 38 shows an end view of the endotracheal tube and stylet;

FIG. 39 shows a rear elevational view of the endotracheal tube and stylet of the present invention;

FIG. 40 shows a close-up cross-sectional view of the distal tip end of an endotracheal tube with the stylet of the present invention residing therein to articulate the distal tip of the endotracheal tube;

FIG. 41 is a cross-section of a patient showing the desire range of motion to articulate the tip of an endotracheal tube of the present invention to guide it into the trachea of the patient;

FIG. 42 shows actuation of the lever to cause upward articulation of the tip of the stylet of the present invention to, in turn, articulate the tip of the endotracheal tube upwards;

FIG. 43 shows the lever in a neutral position to cause positioning of the tip of the stylet of the present invention to be in a neutral position thereby positioning the tip of the endotracheal tube in a neutral position; and

FIG. 44 shows actuation of the lever to cause downward articulation of the tip of the stylet of the present invention to, in turn, articulate the tip of the endotracheal tube downwards.

### DESCRIPTION OF THE INVENTION

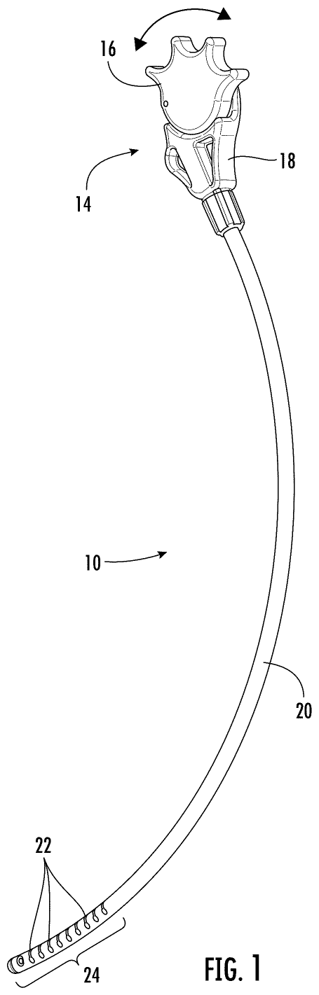

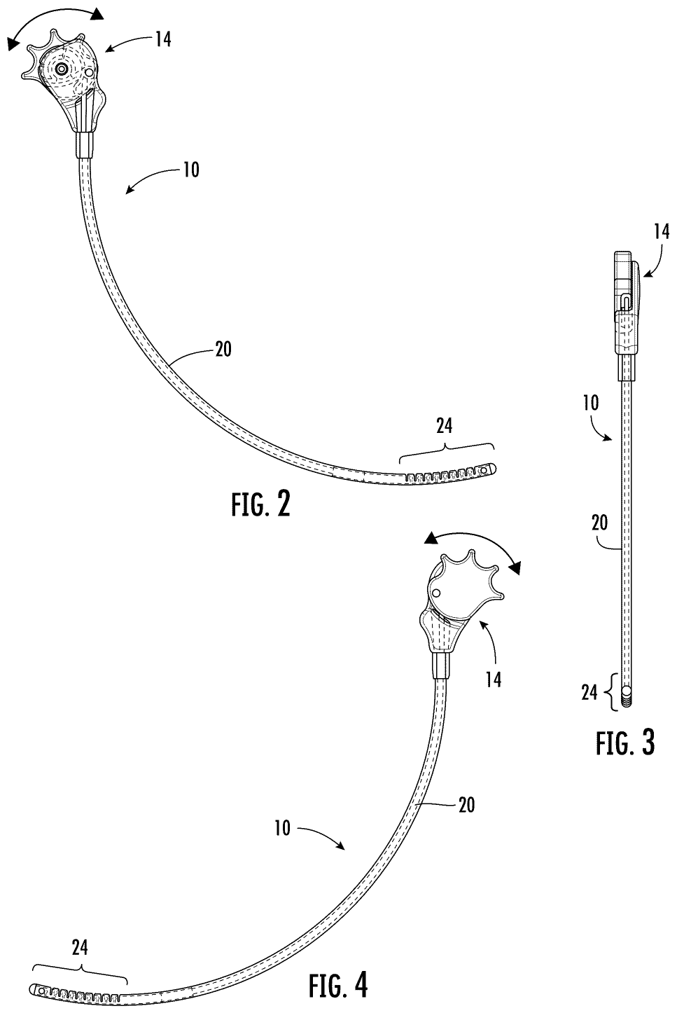

In view of the foregoing, the present invention provides a new, improved, and novel articulating stylet 10 to steer an ETT 12 during intubation. Referring first to FIG. 1, a perspective view of the articulating stylet 10 of the present invention is shown to include a handle 14 with a lever 16, pivot shroud 18 and outer sleeve 20 with a number of tessellations 22 at the distal tip 24 thereof so articulation of the stylet 10 is focused in that tip region 24. In general, pivoting of the lever 16 causes a wire core 26 (not shown in FIG. 1 but discussed and shown below) to move inside the outer sleeve 20 to translation movement to the tip 24 of the stylet 10 which, in turn, is translated to an ETT 12 residing thereover. FIGS. 2-4 show various general views of the articulating stylet 10 of the present invention residing in an ETT.

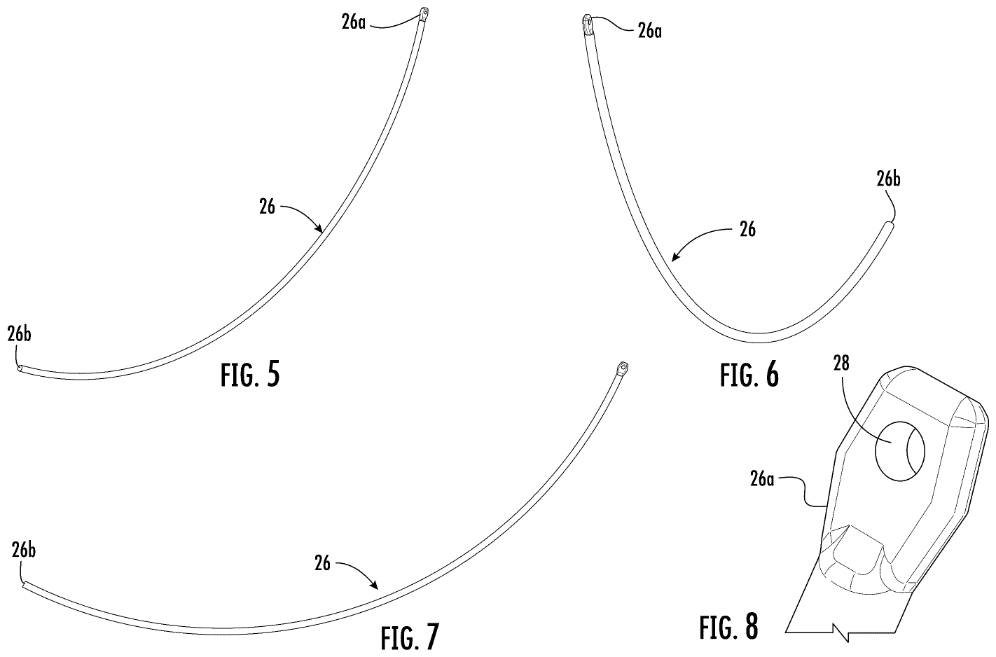

Turning now to FIGS. 5-8, details of the wire core 26 is shown. The wire core 26 includes a proximal end 26a and a distal end 26b where the proximal end 26a preferably includes an eyelet 28 for a Clevis type interconnection of the proximal end 26a of the wire core 26 to the lever 16, as will be discussed in detail below. FIG. 8 shows a close-up view of the eyelet 28 for the Clevis type interconnection. The distal end 26b of the wire core 26 is insertable into a bore 32 of the inner spine 30, as will be discussed below. The wire core 26 is preferably a metal wire but may be made of plastic and may be of other configurations. The wire core 26 is preferably 0.105″ in diameter and 12.25″ in length and made of 420 stainless steel but may be different sizes and made of different materials.

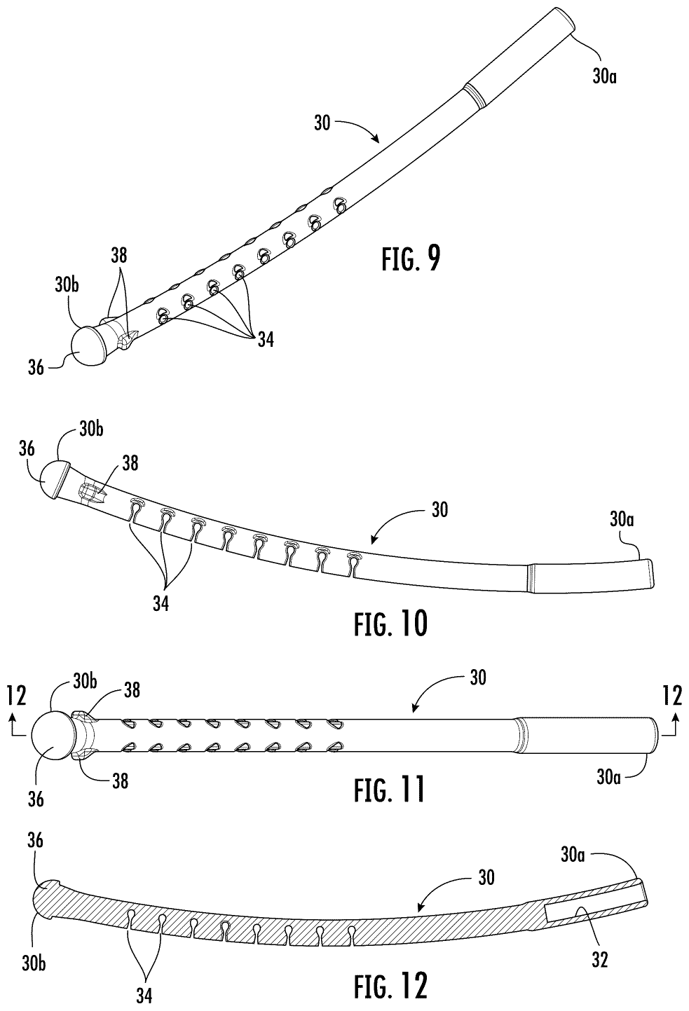

FIGS. 9-12 show different view of the inner spine member 30 of the present invention. FIG. 9 shows a perspective view of the inner spine 30 while FIG. 10 is a side elevational view and FIG. 11 is a top view. FIG. 12 shows a cross-section view through the line 12-12 of FIG. 11. The inner spine 30 includes an expanded end with a central bore 32 therein to receive the distal end 26b of the wire core 26. The wire core 26 is inserted into the bore 32 in the inner spine member 30 and is retained in place by friction, and the like. Thus, the inner spine member 30 is attached to the free distal end 26b of the wire core 26.

The inner spine member 30 includes a number of wedged tessellations 34 at the distal end 30b thereof to limit deflection of the tip 24 of the stylet 10, such as providing a limit of motion of 80 degrees.

The shape of inner spine member 30 is preferably extended to accommodate buy-out of material limitation but the distance between the tessellations 34 and wire core attachment is preferably as minimal distances as possible. Further the tessellations 34 preferably include additional material added to the root of the tessellations 34 for added durability. The distal free end 30b of the inner spine member 30 includes a dome member 36 so that the diameter of the distal tip 30b at the dome 36 closely matches the diameter of the outer sleeve 20 to provide a smooth overall outer surface of the stylet 10 of the present invention. Also, the inner spine member 30 includes a catch feature 38 on the distal end 30b of the inner spine member 30. The catch feature 38 is preferably two ramped members on opposing sides of the inner spine member 30 to each other. This enables the outer sleeve 20 to be secured to the inner spine member 30 without bonding. For assembly, the wire core 26 is inserted into the bore 32 of the inner spine member 30 and then the wire core 26 is inserted into the distal end 20b of the outer sleeve 20 so that the catch features 38 on the inner spine member 30 snaps into opposing holes 40 at the distal end 20b of the outer sleeve 20 to secure the inner spine member 30 to the inside of the outer sleeve member 20. Still further, the distal end 30b of the inner spine member 30 flares slightly outward to provide an improved fit within the outer sleeve member 20 when the inner spine member 30 is inserted into the outer sleeve 20 for attachment via the catch members 38. Details of the outer sleeve 20 and how it interconnects to the inner spine member 30 will be discussed in detail below.

The inner spine member 30 is preferably made of plastic but can be made of other materials, such as metal.

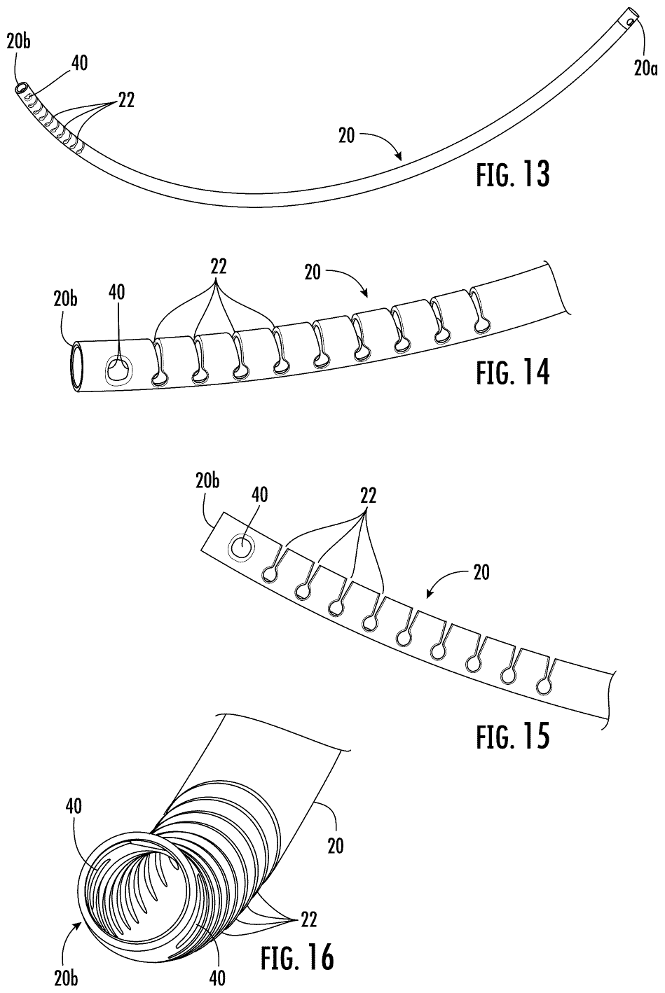

FIGS. 13-16 show further details of the outer sleeve 20 of the articulating stylet 10 of the present invention. In FIG. 13, a perspective view of the outer sleeve 20 of stylet 10 of FIG. 1 of the present invention is shown. The outer sleeve 20 is tubular in configuration and is preferably made of Teflon but may be made of other materials. The outer sleeve 20 includes a proximal open end 20a and a distal open end 20b. A number of tessellations 22 are provided over a portion of the free distal end 20b of the outer sleeve 20. FIG. 14 shows s a close-up perspective view of the outer sleeve 20 of FIG. 13 showing the tessellations 22, which are of similar configuration as the tessellations 34 on the inner spine member 30 to help control the direction and amount of deflection of the tip region 24 of the stylet 10 of the present invention. FIG. 15 shows a side elevational view of the outer sleeve 20 of FIG. 13 where the wedge type tessellations 22 can be seen in detail. Such tessellations 22 may use a modified die cut shape to include a reduced sized root portion that is paired with, for example, a 15 degree wedge cut. There may be nine tessellations 22 with 15 degrees for each tessellation with a 135 degree limit, for example. Other configurations with different sizes and number of tessellations 22 may be used and considered within the scope of the present invention.

Further, FIG. 16 shows a close-up end perspective view of the distal end 20b of the outer sleeve 20 of FIG. 13 to illustrate the tubular configuration of the outer sleeve 20 so it can receive the connected wire core 26 and inner spine member 30 through the open distal end 20b of FIG. 16.

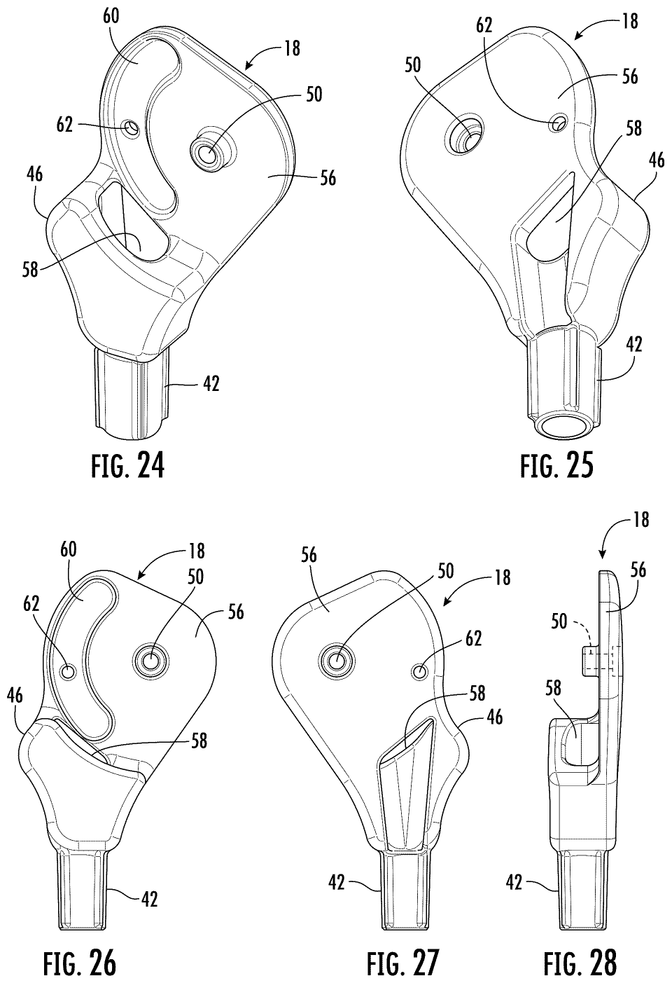

In FIGS. 14 and 15, two opposing holes 40 are provided at the distal end 20b of the outer sleeve 20 for receiving the catch ramps 38 of the inner spine member 30. Thus, the outer sleeve 20 provides an indexing snap fit punch out holes 40 to receive the catches 38. Moreover, the preferably two catch ramps 38 snap into the respective opposing holes 40 in the outer sleeve member 20. The opposing proximal end 20a of the outer sleeve 20 is bonded to a cylindrical tube portion 42 of the pivot shroud 18, as seen in FIGS. 24-28.

The outer sleeve 20 is preferably Teflon tubing that has a natural curve/bias to avoid additional forming to be most compatible with the anatomy of a patient's throat. The Teflon tubing may be 6 mm OD×5 mm ID and may be 310 mm long. While these are preferred dimension and materials. Other sizes and materials may be used.

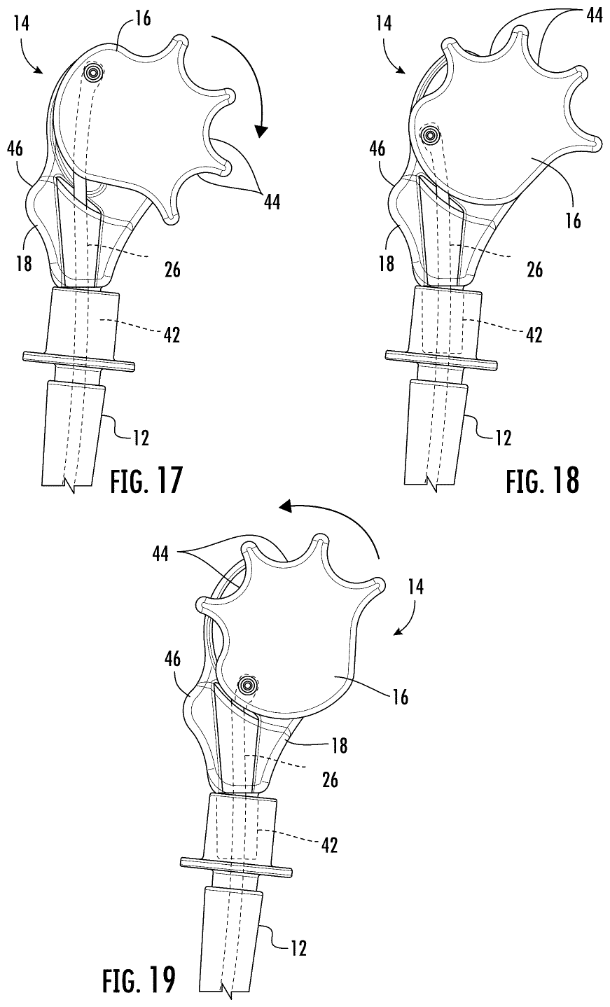

FIGS. 17-19 show partial views of the lever portions 16 of the handle portion 14 of the articulating stylet 10 of the present invention where a user manipulateable lever 16 is pivotally mounted to a pivot shroud 18 where the wire core 26 is pivotally secured to an inner surface of the lever 16 via a Clevis type connection. In FIG. 17, a side elevational view of the lever 16, mounted on a pivot shroud 18, is shown in a upward position corresponding to an upwardmost position of the tip region 24 of the stylet 10 of the present invention. In FIG. 18, a side elevational view of the lever 16 in a neutral position corresponding to a neutral position of the tip region 24 of the stylet 10 of the present invention. FIG. 19 shows a side elevational view of the lever 16 in a downward position corresponding to a downwardmost position of the tip region 24 of the stylet 10 of the present invention. The proximal free end 26a of the wire core 26 is affixed at a Clevis connection point near the periphery of the pivoting lever 16. The pivot shroud 18 rotatably/pivotally receives the lever 16, which has as a number of finger notches 44 to facilitate gripping to provide additional leverage for the user during adjustment and articulation. The pivot shroud 18 has a shoulder 46 to assist the user in rotating the lever 16 to enable precise control of the movement of the lever 16 movement to, in turn, control movement of the wire core 26. The finger notches 44 provide an enhanced gripping surface for added comfort and control for the user as well as the ability for expanded range of actuation of the lever 16 for further tip articulation. For ease of illustration, the most proximal portion of an intubation tube 12, which is typically 8 mm in diameter, is shown releasably interconnected to a bottom cylindrical portion 42 of the pivot shroud 18, as will be described below.

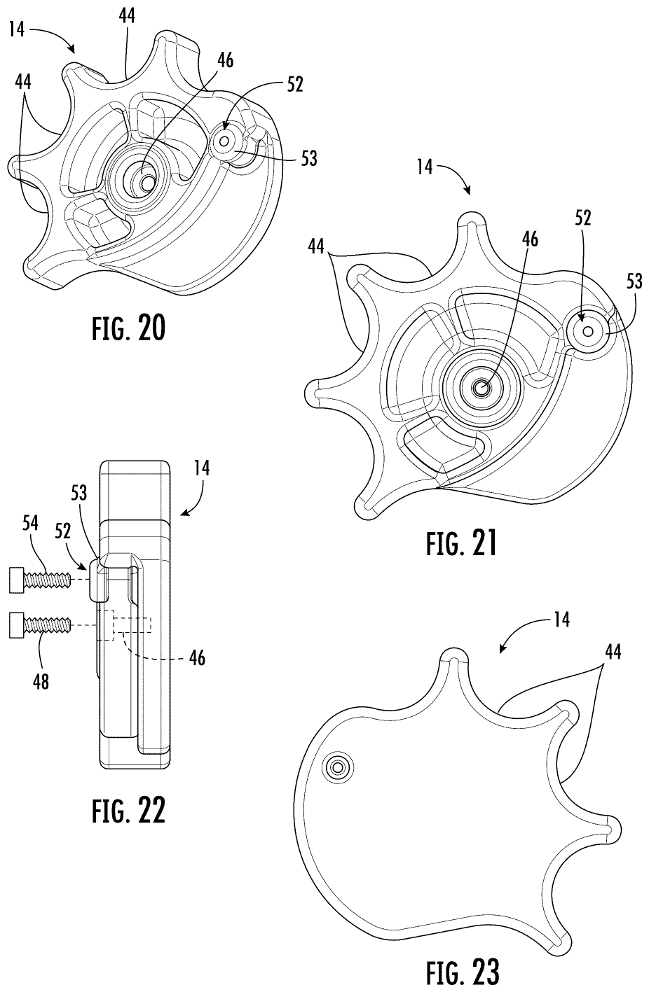

Referring now to FIGS. 20-23, details of the lever 16 is shown while FIGS. 24-28 show details of the pivot shroud 18 onto which the lever 16 is pivotally mounted via bore 50 in the pivot shroud 18. In FIGS. 20 and 21, an inner portion of the lever 16 is shown to include a central pivot bore 46 which terminates inside the lever 16. A fastener 48, such as a pin, is routed through the bore 50 in the pivot shroud 18 and into pivot bore 46 in the pivot shroud 18 and into engagement with the lever 16. This permits the lever 16 to rotate/pivot about the fastener 48 in the center of the lever 16. Thus, the lever 16 rotates like a control dial.

The lever 16 also has a boss with a U-shaped Clevis type mount structure 52 for interconnection to the eyelet 28 of the proximal end 26a of the wire core 26, as seen in FIGS. 5-8. The proximal end 26a of the wire core 26 fits inside the U-shaped Clevis mount 52 and is secured in place with a pin 54. Thus, the rotation of the lever 16, with the assistance of the finger notches 44, causes the wire core 26 to move within the outer sleeve 20 to thereby articulate the distal end 20b thereof because the distal end 26b of the wire core 26 is fixed to the outer sleeve 20 via the inner spine member 30, namely, the catch ramps 38 to the respective holes 40 in the outer sleeve 20 at the distal end 20b thereof.

FIGS. 24-28 show details of the pivot shroud 18 which carries the lever 16 so it can provide the rotation and pivot action to move the wire core 26. More specifically, FIG. 24 is a front perspective view of the pivot shroud 18 of the stylet 10 of the present invention while FIG. 25 is a rear perspective view of the pivot shroud 18 of FIG. 24, FIG. 26 is a front view of the pivot shroud 18 of FIG. 24, FIG. 27 is a rear view of the pivot shroud 18 of FIG. 24 and FIG. 28 is a side elevational view of the pivot shroud 18 of FIG. 24. The pivot shroud 18 includes a back plate 56 with the pivot bore 50 therethrough to provide main pivotal mounting of the lever 16 thereto. The pivot bore 50 is preferably recessed as can be seen in FIGS. 25 and 27. The wire core 26 is routed up through the open cylinder intubation neck 42 interface and through the opening 58 at the bottom of the pivot shroud 18 whereby the wire core 26 passes through the cylinder 42 and then up to the aforesaid connection to the lever 16, as shown in FIGS. 17-19.

Referring to FIGS. 24 and 26 in particular, a Clevis pivot travel recess 60 is provided within the face of back plate 56 of the pivot shroud 18. A spine pivot access hole 62 is provided to permit securing a fastener 54 to attach the proximal end 26a of the wire core 26 to the lever for the Clevis pivot interconnection 52. In other words, the access hole 62 enables access to both sides of the wire core 26 after it is routed through the intubation neck interface 42 and is residing between the pivot shroud 18 and the lever 16. The boss 53 of the Clevis connection 52, as seen in FIGS. 20-22, rides within the travel recess 60 in the back plate 56 of the pivot shroud 18 where the extent of rotational pivot travel is thereby limited by the length of the arcuate travel recess 60. The extent of the travel recess is preferably less than 180 degrees but more than 90 degrees to control the amount of deflection of the tip region 24 of the stylet 10 to avoid damage to it and also to avoid excessive deflection that might cause injury to the patient.

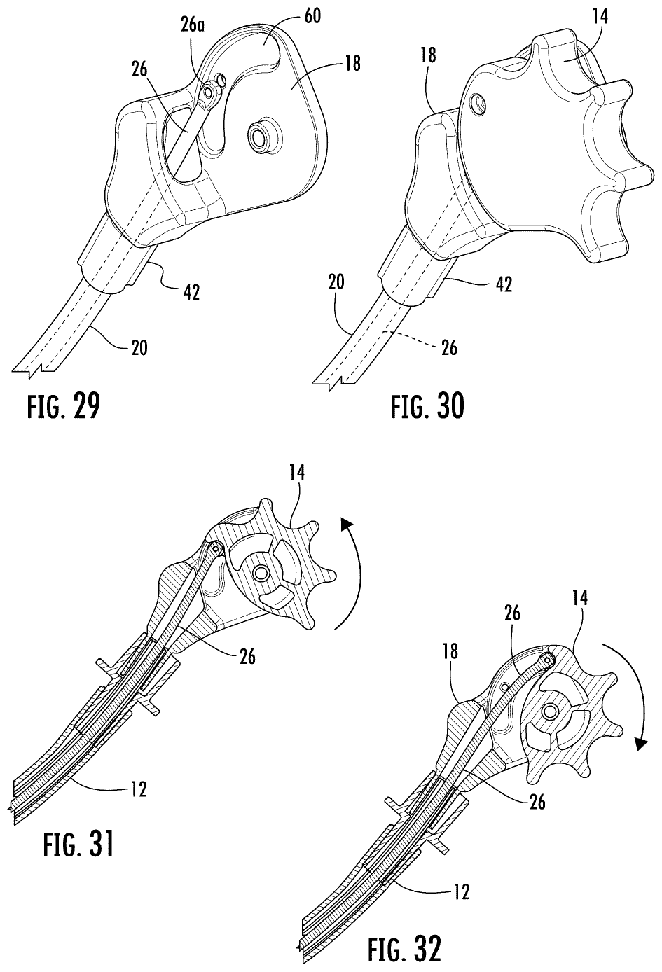

FIGS. 29-32 show additional details of the interconnection of the wire core 26 to the lever 16 and pivot shroud portion 18 of the stylet 10 of the present invention. FIG. 29 shows the wire core 26 emanating up through the intubation neck interface 42 where the neck is bonded to the outer sleeve 20. The handle 14 is not shown for illustration purposes. In FIG. 30, the lever 16 is secured to the proximal eyelet end 26a of the wire core 26 as described above. Once secured in place the proximal end 26a of the wire core 26 is attached to the lever 16 so when the lever 16 is rotated like a dial, the wire core 26 moves within the outer sleeve 20. In FIG. 32, the rotation position of the lever 16 is such that the connection point of the proximal end 26a of the wire core 26 is in a neutral or rest position, namely, approximately in the middle of the path of the travel recess 60. On the other hand, FIG. 32 shows the lever 16 rotated as clockwise as possible where it meets the uppermost edge of the travel recess 60 thereby defining the upwardmost position of the wire core 26 corresponding to the uppermost position of the articulating stylet 10, as shown in FIG. 17, for example.

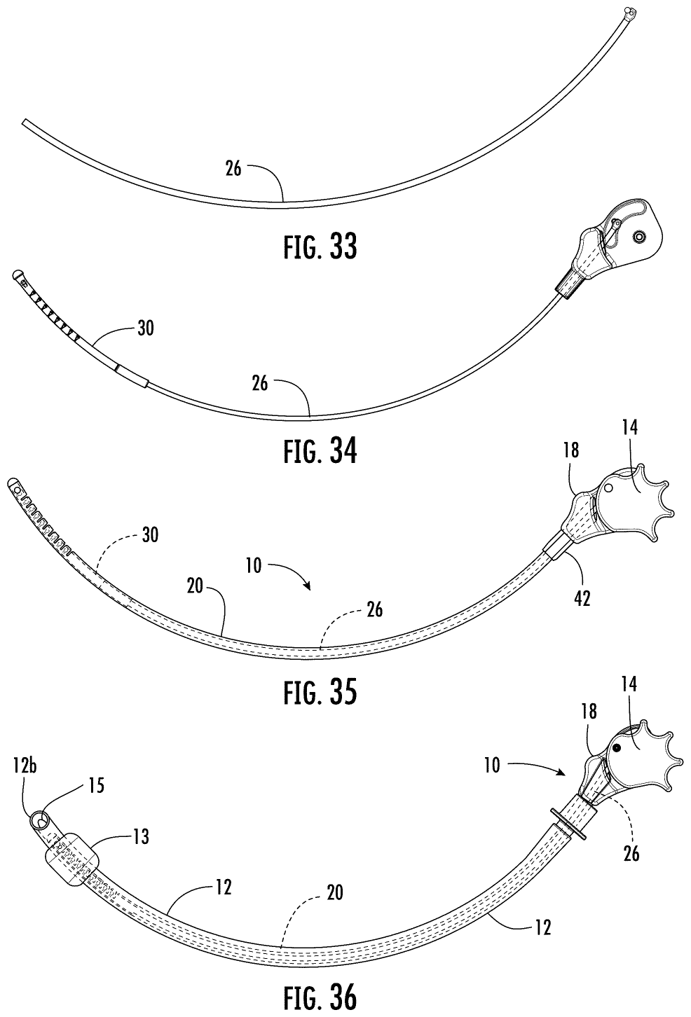

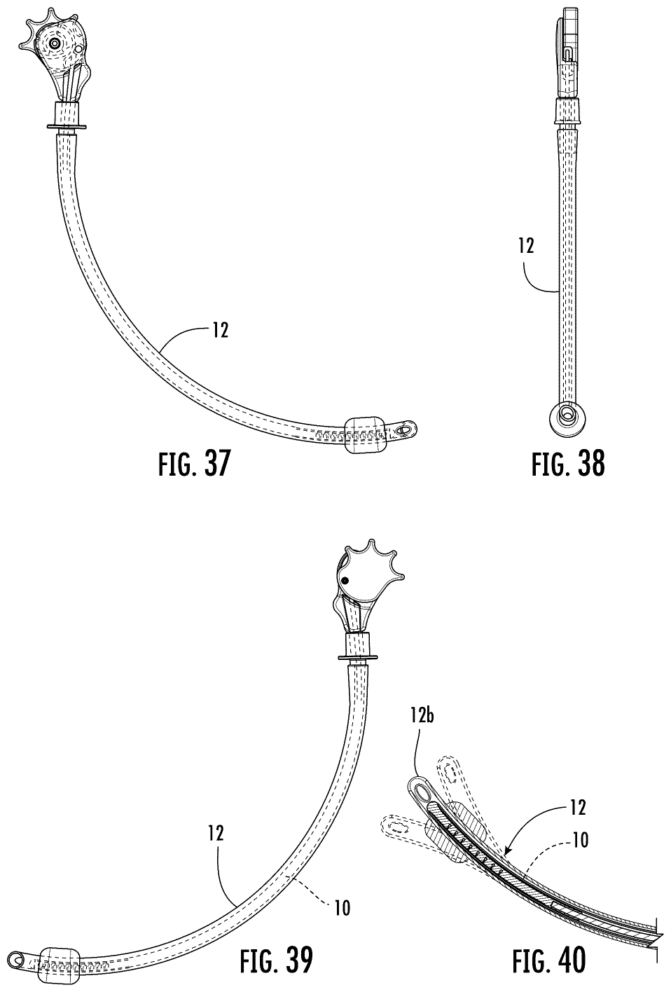

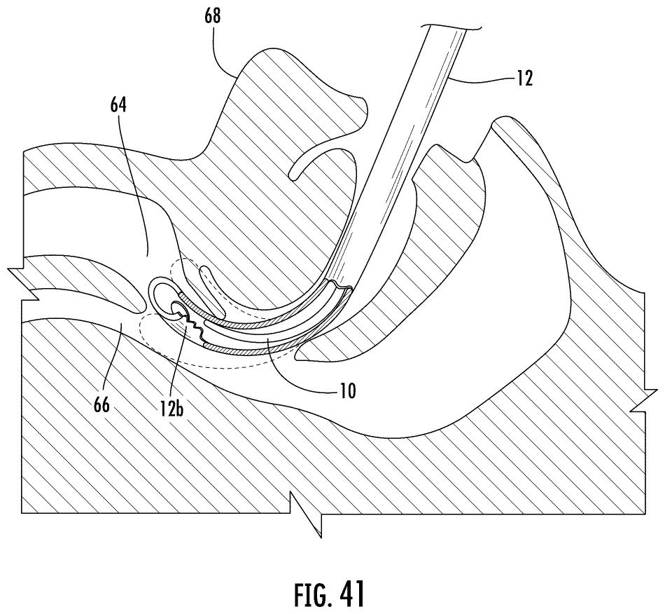

FIGS. 33-36 provides an overview of the different components to show the construction of the articulating stylet 10 of the present invention. First, FIG. 33 shows the wire core 26 and FIG. 34 shows the inner spine 30 member attached to the distal end of the wire core 26. FIG. 35 shows the outer sleeve 20 residing over the inner spine 30 and wire core 26 to provide a completely assembled stylet 10 of the present invention. FIG. 36 shows an ETT 12, with cuff 13 and Murphy eye 15, routed over the stylet 10 of the present invention in preparation for use in an intubation procedure. FIGS. 37-39 show additional views of an ETT 12 mounted on the stylet 10 of the present invention in preparation for use in an intubation procedure. FIG. 40 shows a close-up cross-sectional view of the distal tip end 12b of an ETT with the stylet 10 of the present invention residing therein to articulate the distal tip 12b of the ETT 12. The broken lines show a sample range of deflection of the ETT 12 that can be created due to the articulation of the stylet 10 residing therein. The force created by the stylet 10 of the present invention, with its unique Clevis connection 52 with lever 16 and pivot shroud 18, provides sufficient force that can be imparted to the ETT 12 itself to articulate it as desired.

FIG. 41 is a cross-section of a patient 68 showing the desire range of motion to articulate the tip 12b of an endotracheal tube 12 of the present invention to guide it into the trachea 64 of the patient 68. As can be seen, it is desirous to articulate the distal tip 12b of the ETT 12, via the stylet 10, to ensure that the ETT 12 is steered into the trachea 64 of the patient and not the esophagus 66. Similar to the broken lines of FIG. 40, the broken lines in FIG. 41 show the possible range of deflection to successfully guide the ETT 12 in real time during the intubation procedure.

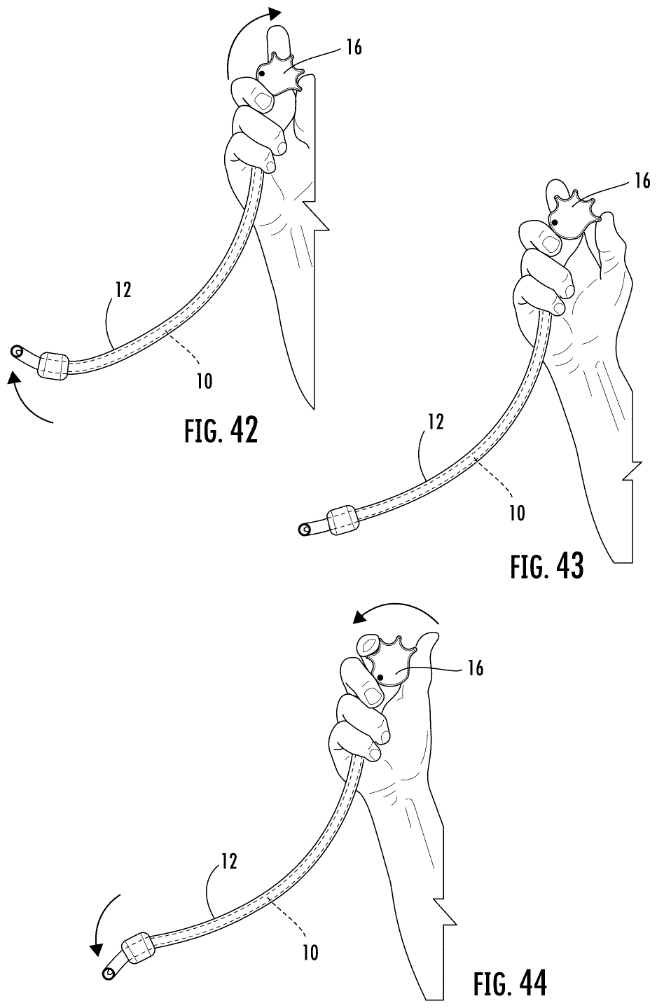

FIG. 42 shows actuation of the lever 16 to cause upward articulation of the tip region 24 of the stylet 10 of the present invention to, in turn, articulate the tip 12b of the ETT 12 upwards while FIG. 43 shows the lever 16 in a neutral position to cause positioning of the tip region 24 of the stylet 10 of the present invention to be in a neutral position thereby positioning the tip 12a of the ETT 12 in a neutral position. Lastly, FIG. 44 shows actuation of the lever 16 to cause downward articulation of the tip region 24 of the stylet 10 of the present invention to, in turn, articulate the tip 12b of the ETT 12 downwards. Thus, the finger actuated rotation of lever 16 moves the wire core 26 within the stylet 10 to articulate it as desired with precision to, in turn, guide the tip 12b of the ETT 12 itself for proper location of the ETT 12 in the patient 68.

It would be appreciated by those skilled in the art that various changes and modifications can be made to the illustrated embodiments without departing from the spirit of the present invention. All such modifications and changes are intended to be covered by the appended claims.

What is claimed is:

1. An articulating stylet to facilitate insertion of an endotracheal tube, comprising:
a core member having a proximal end and a distal end;
an inner spine, having a proximal end and a distal end; the distal end of the core member being connected to the proximal end of the inner spine;
a hollow outer sleeve having a proximal end and a distal end and defining a longitudinally running lumen therein; the distal end of the inner spine being connected to the distal end of the hollow outer sleeve;
a pivot shroud comprising the proximal end of the handle which is securely fastened to the hollow outer sleeve;
a lever pivotally attached to the pivot shroud at a pivot point thereon; the proximal end of the core member being attached to the pivot point of the lever;
wherein pivoting actuation of the lever relative to the pivot shroud/handle/outer sleeve complex moves the core member within the articulating stylet of the present invention The core member thereby deflects the distal end of the hollow outer sleeve with sufficient force from the pivoting and mechanically advantaged actuation of the lever to deflect an endotracheal tube slidably received over the hollow tube.

2. The articulating stylet of claim 1, wherein the core member is a malleable wire.

3. The articulating stylet of claim 1, wherein the core member, pivot shroud and lever are made of plastic or metal.

4. The articulating stylet of claim 1, wherein the inner spine includes tessellations in the region of the distal end of the inner spine thereby limiting deflection of the inner spine.

5. The articulating stylet of claim 4, wherein the distal end of the inner spine further includes a domed distal tip that has a diameter that is substantially the same as an endotracheal tube.

6. The articulating stylet of claim 1, wherein the outer sleeve includes tessellations in the region of the distal end of the inner spine thereby limiting deflection of the inner spine.

7. The articulating stylet of claim 5, wherein a diameter of domed distal tip is substantially the same as the diameter of the endotracheal tube.

8. The articulating stylet of claim 1, wherein the outer sleeve is made of Teflon.

9. The articulating stylet of claim 1, wherein the core member is attached to the lever at a point other than the pivot point.

10. The articulating stylet of claim 9, wherein the core member includes an eyelet on the proximal end thereof and is attached to a boss of the lever by a clevis connection.

11. The articulating stylet of claim 10, wherein the pivot shroud includes a partial arcuate travel recess defining a range of travel of the lever; the boss travels within the travel recess thereby defining the amount and location of the pivot shroud.

12. The articulating stylet of claim 1, wherein the lever includes at least one notch which is configured and arranged to releasable receive a user's finger therein.

13. The articulating stylet of claim 1, wherein pivot of the lever in a first direction causes the core member to deflect in a first direction and rotation of the lever in a second direction causes the core member to deflect in a second direction that is opposite to the first direction.

14. The articulating stylet of claim 1, wherein the inner spine further comprises at least one male engagement member near the distal end of the inner spine; the outer sleeve including at least one female engagement member near the distal end of the outer sleeve; the at least one male member respectively engaging with at least one female member to secure the inner spine within the distal end of the outer sleeve.

15. A method for intubating a patient, comprising the steps of:
providing an articulating stylet to facilitate insertion of an endotracheal tube, having:
a core member having a proximal end and a distal end;
an inner spine, having a proximal end and a distal end; the distal end of the core member being connected to the proximal end of the inner spine;
a hollow outer sleeve having a proximal end and a distal end and defining a longitudinally running lumen therein; the distal end of the inner spine being connected to the hollow outer sleeve;
a pivot shroud secured to the proximal end of the hollow outer sleeve;
a lever pivotally attached to the pivot shroud at a pivot point thereon; the proximal end of the core member being attached to the pivot point of the base plate;
providing an endotracheal tube having a proximal end and a distal end;
mounting the articulating stylet into the proximal end of an endotracheal tube;
inserting the articulating stylet and endotracheal tube residing therein into a patient's mouth;
actuating the lever while the articulating stylet and endotracheal tube is being inserted into the patient's mouth to deflect the articulating stylet and endotracheal tube to thereby assist in navigation of the articulating stylet and endotracheal tube at the same time;
locating the articulating stylet and endotracheal tube where desired in a patient; and
removing the articulating stylet from endotracheal tube thereby leaving the endotracheal tube in the patient in preparation for delivery of oxygen into the patient via the endotracheal tube.

16. An articulating stylet to facilitate insertion of an endotracheal tube, comprising:
a malleable metal wire core member having a proximal end and a distal end;
an inner spine, having a proximal open end and a distal end; the distal end of the core member being connected to the proximal open end of the inner spine by friction fit; the inner spine including at least male fastening member thereon; the inner spine including a number of tessellations at it distal end;
a hollow outer sleeve having a proximal end and a distal end and defining a longitudinally running lumen therein; the hollow outer sleeve including a number of tessellations at its distal end; the hollow outer sleeve having at least one complementary female fastening member; the inner spine being connected to the distal end of the hollow outer sleeve by interconnection of at least one male member to the at least one female members;
a pivot shroud secured to the proximal end of the hollow outer sleeve; the pivot shroud defining a travel recess;
a lever pivotally attached to the pivot shroud at a pivot point thereon; the proximal end of the core member being attached to the lever via a boss on the lever; the boss traveling within the confines of the travel recess;
wherein pivoting actuation of the lever relative to the pivot shroud moves the core member within the articulating stylet of the present invention; the core member thereby deflects the distal end of the hollow outer sleeve to thereby deflect an endotracheal tube slidably received over the hollow tube.
